(19) United States
(12) Patent Application Publication
(10) Pub. No.: US 2025/0391554 A1
(43) Pub. Date: Dec. 25, 2025

(54) SYSTEMS, METHODS AND APPARATUS FOR NEURO CONDITIONING A BREATHING RESPONSE

(71) Applicant: Rakesh Mohan Lal, Lexington, MA (US)

(72) Inventor: Rakesh Mohan Lal, Lexington, MA (US)

(21) Appl. No.: 18/748,598

(22) Filed: Jun. 20, 2024

(51) Int. Cl. G16H 40/63; A61B 5/00; G16H 20/40

(52) U.S. Cl. CPC G16H 40/63; A61B 5/6844; A61B 5/74; G16H 20/40

(57) ABSTRACT

Systems, methods and apparatus are provided through which in some aspects a breathing response is neuro-conditioned and thereafter used in eliciting a breathing response upon detection of a reduction in breathing or a cessation of breathing. In one aspect, a body worn breathing neuro-conditioning device includes a sensor that senses a set of one or more physiological signals correlated with breathing, a stimulator operable to apply a physical stimulus, a processor, and a set of machine readable instructions which cause to processor to operate in a training more or a monitoring mode, wherein, when operating in the training mode, the processor triggers the stimulator to apply the stimulus periodically, and wherein when operating in the monitoring mode, the processor detects a reduction in or a cessation of breathing and further triggers the stimulator to apply the physical stimulus when the reduction in or cessation of breathing is detected.

### FIELD OF THE INVENTION

This invention relates generally to systems, methods and apparatus for training to establish neuro conditioning of a breathing response to a stimulus when breathing rate reduces or ceases.

### BACKGROUND OF THE INVENTION

Breathing is a fundamental function of the human body and is required to sustain life. Breathing enables intake of oxygen and expulsion of carbon dioxide and other gases produced by the body as part of the respiration process. Reduction in breathing rate and/or volume can lead to medical issues such as hypoxia and hypoxemia, and if such reductions in breathing continue, it can lead to tissue and brain damage and eventually death. Cessation of breathing for a prolonged period of time is fatal. Periodic reductions in breathing, such as during sleep, can also have a long term negative effect on health, and can lead to major health issues such as cardiovascular and neurocognitive decline over time.

While the healthy human body is effective at regulating breathing, there are several medical conditions that affect the body's natural ability to initiate and control breathing. Sleep apnea is a significant medical condition that affects hundreds of millions of people globally, which may result in interrupted, reduced or stopped breathing during sleep. The manifestation of sleep apnea may be due to obstructions in the airway during sleep in obstructive sleep apnea (OSA), or due to issues with the central nervous system in central sleep apnea. In either scenario, reduction in breathing results and can lead to long term negative health effects.

Another group of patients at high risk of suffering the consequences of reduced or stopped breathing are those with substance use disorder (SUD). Various substances such as opioids, including morphine, heroin, and fentanyl, interfere with the body's natural breathing mechanisms and when taken in excess, can significantly reduce or stop breathing putting the patient at significant risk of harm. Clinically, opioid induced respiratory depression (OIRD) is the medical condition resulting from opioid overdose and is known to be very harmful and can lead to death. It is estimated that approximately one hundred thousand deaths occur annually in the US alone due to OIRD. Globally, the figure is much larger and the number of hospitalizations and permanent injury cases, including permanent brain damage, resulting from OIRD is also significantly larger.

For the reasons stated above, and for other reasons stated below which will become apparent to those skilled in the art upon reading and understanding the present specification, there is a need in the art for a system to detect any situations where there is a reduction or cessation of breathing and to trigger a breathing response in these situations.

### BRIEF SUMMARY OF THE INVENTION

The above-mentioned shortcomings, disadvantages and problems are addressed herein, which will be understood by reading and studying the following specification.

In one aspect, a body worn, breathing response neuro-conditioning device includes a sensor operable to continuously sense a set of one or more physiological signals that change as breathing changes, a stimulator operable to apply a stimulus, a processor operable to receive the set of one or more physiological signals and further operable to trigger the stimulator to apply the physical stimulus, and a set of machine readable instructions embodied in non-transitory memory which cause the processor to operate in either a training mode or a monitoring mode, such that (i) when operating in the training mode, the machine readable instructions cause the processor to trigger the stimulator periodically at variable intervals determined by a predetermined set of rules, and such that (ii) when operating in the monitoring mode, the machine readable instructions cause the processor to detect a reduction in the rate of breathing to a predetermined rate value, or a cessation of breathing, and further cause the processor to trigger the stimulator to apply the physical stimulus when the reduction in the rate or the cessation of breathing is detected.

In another aspect, a method for conditioning a breathing response includes determining whether to operate in a training mode or in a monitoring mode, and when operating in the training mode, further includes triggering a stimulator periodically at variable intervals determined by a predetermined set of rules, to apply a stimulus, and when operating in the monitoring mode, further includes sensing a set of one or more physiological signals that change as breathing changes, detecting a reduction in the rate of breathing or a cessation of breathing from the set of one or more physiological signals and triggering the stimulator to apply the physical stimulus when the reduction in the rate of breathing or the cessation of breathing is detected.

Apparatus, systems, and methods of varying scope are described herein. In addition to the aspects and advantages described in this summary, further aspects and advantages will become apparent by reference to the drawings and by reading the detailed description that follows.

### BRIEF DESCRIPTION OF THE DRAWINGS

FIG. 1 is a block diagram of an apparatus to neuro-condition a breathing response using one or more sensors, a stimulator, a processor, and a set of machine readable instructions that cause the sensor to operate either in a training mode and a monitoring mode.

FIG. 2 is a block diagram of an apparatus according to an aspect to neuro-condition a breathing response using one or more sensors wherein some of the sensors include an airflow sensor or an end tidal carbon dioxide sensor, a stimulator, a processor, and a set of machine readable instructions that cause the processor to operate either in a training mode and a monitoring mode.

FIG. 3 is a block diagram of an apparatus according to an aspect to neuro-condition a breathing response using one or more sensors, a stimulator, a processor, a set of machine readable instructions that cause the processor to operate either in a training mode and a monitoring mode, and an on-skin sensor that determines when the apparatus is on the body and when it is not.

FIG. 4 is a block diagram of an apparatus according to an aspect to neuro-condition a breathing response using one or more sensors, a stimulator, a processor, a set of machine readable instructions that cause the sensor to operate either in a training mode and a monitoring mode, and a user interface with instructions to take one or more breaths when the stimulus is felt when the apparatus is operating in training mode.

FIG. 5 is a block diagram of an apparatus according to an aspect to neuro-condition a breathing response using one or more sensors, a stimulator, a processor, a set of machine readable instructions that cause the processor to operate either in a training mode and a monitoring mode, and a physical switch that is read by the machine readable instructions to determine whether to cause the processor to operate in training mode or monitoring mode.

FIG. 6 is a block diagram of an apparatus according to an aspect to neuro-condition a breathing response using one or more sensors, a stimulator, a processor, a set of machine readable instructions that cause the processor to operate either in a training mode and a monitoring mode, and a communication system to receive a message representing either the training mode or the monitoring mode, and further operable to notify an external device when the stimulator is triggered when operating in monitoring mode.

FIG. 7 is a flowchart of a method to neuro-condition a breathing response by determining when to operate in a training and when to operate in a monitoring mode, setting the operating mode, and periodically triggering a stimulator when in training mode, and sensing physiological signals, detecting reduction or cessation of breathing, and triggering the stimulator when in monitoring mode.

FIG. 8 is a flowchart of a method to neuro-condition a breathing response by determining when to operate in a training and when to operate in a monitoring mode, setting the operating mode, and periodically triggering a stimulator when in training mode, and sensing physiological signals, detecting reduction or cessation of breathing, and triggering the stimulator at increasing levels based on the number of times the reduction or cessation of breathing is detected when in monitoring mode.

FIG. 9 is a flowchart of a method to neuro-condition a breathing response by determining when to operate in a training and when to operate in a monitoring mode, setting the operating mode, and periodically triggering a stimulator and providing instructions on a user interface to take one or more breaths when in training mode, and sensing physiological signals, detecting reduction or cessation of breathing, and triggering the stimulator when in monitoring mode.

FIG. 10 is a flowchart of a method to neuro-condition a breathing response by receiving an operating mode, limited to training or monitoring modes, either wirelessly or electronically through an internal wired system, selecting to operate in a training or monitoring mode, and periodically triggering a stimulator when in training mode, and sensing physiological signals, detecting reduction or cessation of breathing, and triggering the stimulator when in monitoring mode.

FIG. 11 is a flowchart of a method to neuro-condition a breathing response by determining when to operate in a training and when to operate in a monitoring mode, setting the operating mode, and periodically triggering a stimulator when in training mode, and sensing physiological signals, detecting reduction or cessation of breathing, triggering the stimulator, and notifying an external system of the triggering of the stimulator when in monitoring mode.

### DETAILED DESCRIPTION OF THE INVENTION

In the following detailed description, reference is made to the accompanying drawings that form a part hereof, and in which is shown by way of illustration specific aspects which may be practiced. These aspects are described in sufficient detail to enable those skilled in the art to practice the aspects, and it is to be understood that other aspects may be utilized and that logical, mechanical, electrical and other changes may be made without departing from the scope of the aspects. The following detailed description is, therefore, not to be taken in a limiting sense.

The detailed description is divided into four sections. In the first section, a system level overview is described. In the second section, apparatus of aspects are described. In the third section, aspects of methods are described. Finally, in the fourth section, a conclusion of the detailed description is provided.

### System Level Overview

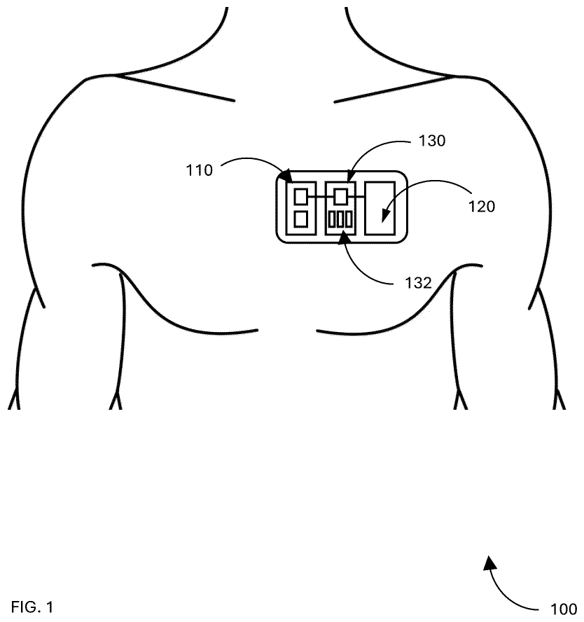

FIG. 1 is a block diagram of a body worn apparatus to neuro-condition a breathing response using one or more sensors, a stimulator, a processor, and a set of machine readable instructions that cause the sensor to operate either in a training mode and a monitoring mode. System 100 solves the need in the art to neuro-condition a breathing response, and to trigger the breathing response when reduced or stopped breathing is detected.

System 100 includes a sensor 110, operable to sense one or more physiological signals that change as breathing changes, a stimulator 120, operable to apply a physical stimulus, a processor 130, operable to receive the set of one or more physiological signals and further operable to trigger the stimulator to apply the physical stimulus, and a non-transitory memory component 132, embodying a set of machine readable instructions which cause the process to operate in either a training mode or a monitoring mode, such that when operating in the training mode, the machine readable instructions cause the processor to trigger the stimulator periodically at variable intervals determined by a predetermined set of rules, and such that when operating in the monitoring mode, the machine readable instructions cause the processor to detect a reduction in the rate of breathing to a predetermined rate value, or a cessation of breathing, and further cause the processor to trigger the stimulator to apply the physical stimulus when the reduction in the rate or the cessation of breathing is detected.

Component 110 solves the need in the art to sense one or more physiological signals that are related to breathing and change when breathing changes.

Component 120 solves the need in the art to apply a physical stimulus when it is required.

Components 130 and 132 solve the need in the art to receive the set of one or more physiological signals, and further solves the need, when operating in a training mode, to apply the stimulus periodically at variable intervals determined by a predetermined set of rules by triggering the stimulator 120 so as to neuro-condition the user to breathe in response to the physical stimulus. Components 130 and 132 further solve the need in the art, when operating in a monitoring mode, to detect a reduction in breathing or a cessation of breathing from the physiological signals, and to apply the stimulus by triggering the stimulator 120 when the reduction in breathing or the cessation of breathing is detected.

The system level overview of the operation of an aspect is described in this section of the detailed description. The sensor continuously senses the one or more physiological signals that are correlated with breathing and as a result change when breathing changes, and which are sent to the processor.

The processor receives the set of one or more physiological signals and when operating in monitoring mode, analyzes these signals to determine whether a reduction in breathing or a cessation in breathing is detected. If such conditions are detected, the processor triggers the stimulator to apply a stimulus, thereby eliciting a breathing response from the user of the device, thus restoring breathing either completely or partially, thereby preventing or delaying the onset of any negative health effects of reduced breathing or stopped breathing.

In order to train or neuro-condition the user of the device to breath in response to the applied stimulus, the processor may operate in a training mode that periodically at variable intervals determined by a predefined set of rules, triggers the stimulator to apply the physical stimulus irrespective of whether any reductions in or cessation of breathing is occurring. The user of the device is further instructed to take one or more deep breaths upon feeling the applied physical stimulus. This process is repeated for a period of time in order to neuro-condition the user to breathe in response to the physical stimulus.

While the system 100 is not limited to any particular sensor or sensors, or any particular physiological signals acquired by the sensor or sensors, or any particular stimulator, for sake of clarity a simplified sensor, set of one or more physiological signals that change as breathing changes, stimulator, and processing system, including processor, non-transitory memory and set of machine readable instructions are described.

### Apparatus Aspects

In the previous section, a system level overview of the operation of an aspect was described. In this section, the particular apparatus of such an aspect are described by reference to a series of diagrams.

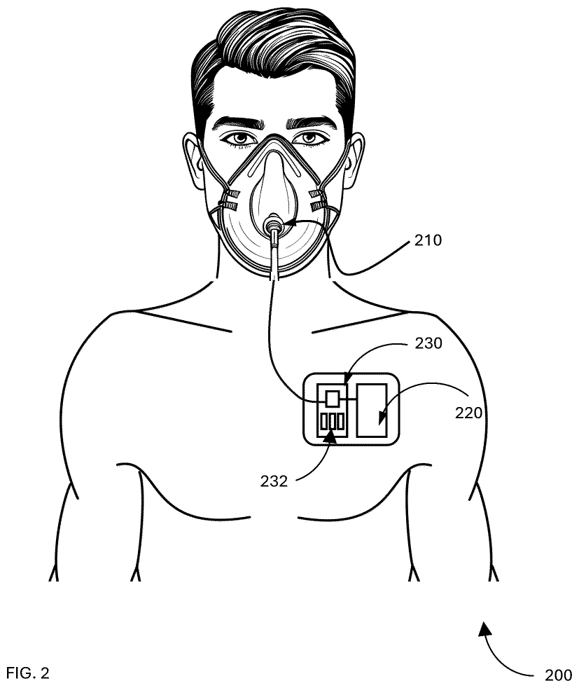

FIG. 2 is a block diagram of an apparatus according to an aspect to neuro-condition a breathing response using one or more sensors wherein some of the sensors include an airflow sensor or an end tidal carbon dioxide sensor, a stimulator, a processor, and a set of machine readable instructions that cause the processor to operate either in a training mode and a monitoring mode.

Apparatus 200 includes a sensor 210 operable to continuously sense a set of one or more physiological signals that change as breathing changes, a stimulator 220 operable to apply a physical stimulus, a processor 230 operable to receive the set of one or more physiological signals and further operable to trigger the stimulator to apply the physical stimulus, and a set of machine readable instructions embodied in non-transitory memory 232 which cause the processor to operate in either a training mode or a monitoring mode, such that (i) when operating in the training mode, the machine readable instructions cause the processor to trigger the stimulator periodically at variable intervals determined by a predetermined set of rules, and such that (ii) when operating in the monitoring mode, the machine readable instructions cause the processor to detect a reduction in the rate of breathing to a predetermined rate value, or a cessation of breathing, and further cause the processor to trigger the stimulator to apply the physical stimulus when the reduction in the rate or the cessation of breathing is detected.

In one aspect, the sensor is worn on the body, such as the chest or wrist and senses a set of one or more physiological signals that are correlated with breathing and therefore change as breathing changes. Such physiological signals may include a photoplethysmography signal sensed using a PPG sensor, a movement signal sensed using an inertial sensor such as an accelerometer or gyroscope, or an auscultation signal sensed using a microphone or other auditory sensor. In another aspect, the sensor is embedded within a mask worn on the face or coupled to a nasal cannula and senses a set of one or more physiological signals that are correlated with breathing and therefore change as breathing changes. Such physiological signals may include an airflow signal sensed with a flow gauge or other sensor, or an end tidal carbon dioxide signal sensed using a CO₂ sensor.

In another aspect, the stimulator may be an actuator that applies a force based physical stimulus resulting from movement within the stimulator. In another aspect, the stimulator may be a vibration motor that applies a vibration based physical stimulus resulting from rapid periodic movement within the stimulator, and in another aspect, the stimulator may be a temperature stimulator that applies a temperature based physical stimulus. In another aspect, the stimulator may be an electrical stimulator that applies an electrical based physical stimulus, and in yet another aspect, the stimulator may be an audio based stimulator that applies a sound based physical stimulus.

In another aspect, the stimulator is further operable to apply the physical stimulus at varying amplitudes, and wherein when the machine readable instructions further cause the processor, when operating in the monitoring mode, to trigger the stimulator to apply the physical stimulus at increasing amplitudes based on the number of times the reduction in the rate or the cessation of breathing is detected.

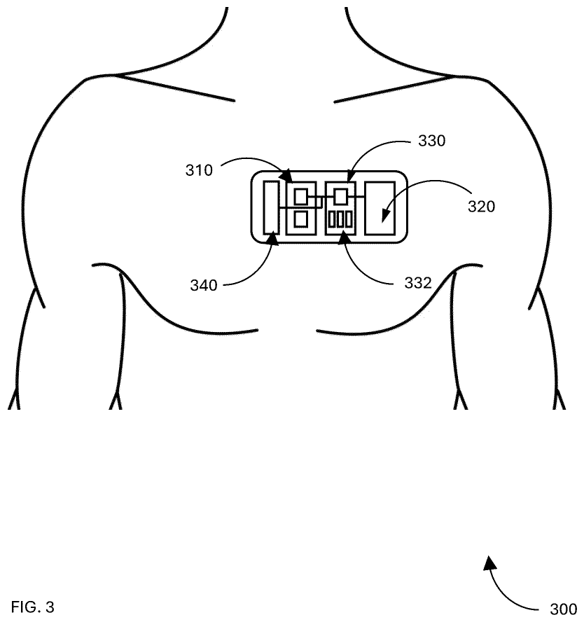

FIG. 3 is a block diagram of an apparatus according to an aspect to neuro-condition a breathing response using one or more sensors, a stimulator, a processor, a set of machine readable instructions that cause the processor to operate either in a training mode and a monitoring mode, and an on-skin sensor that determines when the apparatus is on the body and when it is not.

Apparatus 300 includes a sensor 310 operable to continuously sense a set of one or more physiological signals that change as breathing changes, a stimulator 320 operable to apply a physical stimulus, a processor 330 operable to receive the set of one or more physiological signals and further operable to trigger the stimulator to apply the physical stimulus, an on-skin sensor 340 operable to continuously determine when the device is being worn on the body and when it is not on the body, and a set of machine readable instructions embodied in non-transitory memory 332 which cause the processor to operate in either a training mode or a monitoring mode, such that (i) when operating in the training mode, the machine readable instructions cause the processor to trigger the stimulator periodically at variable intervals determined by a predetermined set of rules, and such that (ii) when operating in the monitoring mode, the machine readable instructions cause the processor to detect a reduction in the rate of breathing to a predetermined rate value, or a cessation of breathing, and further cause the processor to trigger the stimulator to apply the physical stimulus when the reduction in the rate or the cessation of breathing is detected, and wherein the machine readable instructions are further operable to cause the processor to trigger the stimulator only when the on-skin sensor determines that the device is being worn on the body.

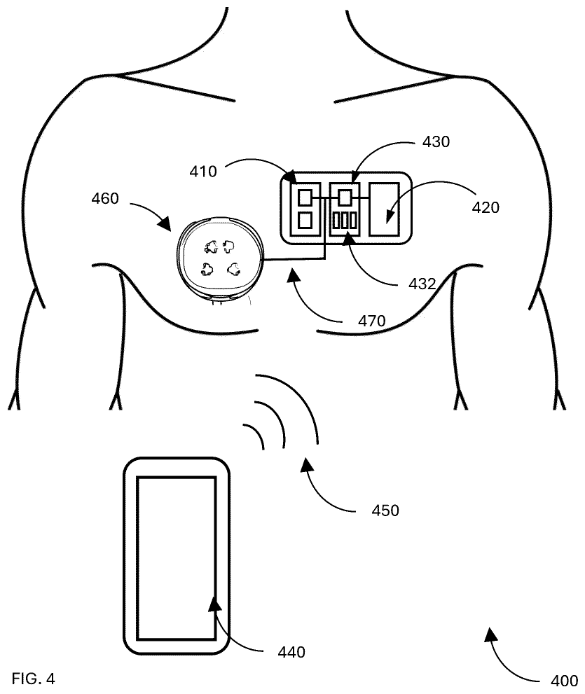

FIG. 4 is a block diagram of an apparatus according to an aspect to neuro-condition a breathing response using one or more sensors, a stimulator, a processor, a set of machine readable instructions that cause the sensor to operate either in a training mode and a monitoring mode, and a user interface with instructions to take one or more breaths when the stimulus is felt when the apparatus is operating in training mode.

Apparatus 400 includes a sensor 410 operable to continuously sense a set of one or more physiological signals that change as breathing changes, a stimulator 420 operable to apply a physical stimulus, a processor 430 operable to receive the set of one or more physiological signals and further operable to trigger the stimulator to apply the physical stimulus, a set of machine readable instructions embodied in non-transitory memory 432 which cause the processor to operate in either a training mode or a monitoring mode, such that (i) when operating in the training mode, the machine readable instructions cause the processor to trigger the stimulator periodically at variable intervals determined by a predetermined set of rules, and such that (ii) when operating in the monitoring mode, the machine readable instructions cause the processor to detect a reduction in the rate of breathing to a predetermined rate value, or a cessation of breathing, and further cause the processor to trigger the stimulator to apply the physical stimulus when the reduction in the rate or the cessation of breathing is detected, and a user interface 440 with instructions to take one or more breaths when the stimulus is felt, when the apparatus is operating in training mode.

In some aspects, the user interface may include a screen 440 that provides visual, text based instructions. In other aspects, the user interface may include a speaker 460 that provides audio based instructions. In some aspects, the user interface may be wirelessly couple with the processor, communicating via radio frequency (RF waves) 450. In other aspects, the user interface may be electrically coupled with the processor via wires 470.

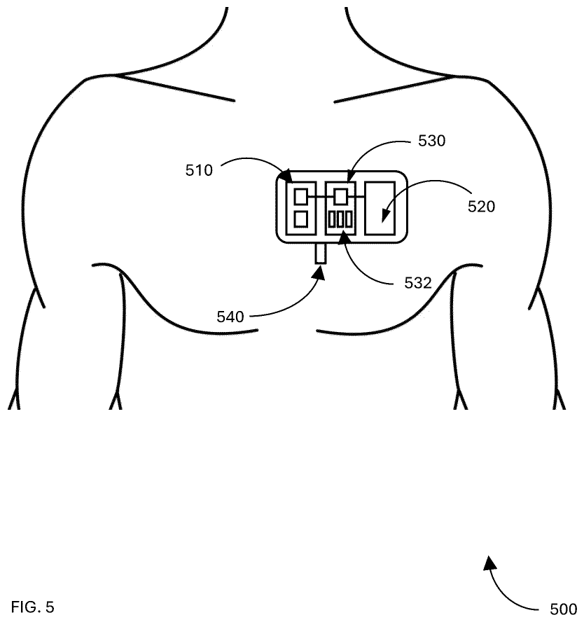

FIG. 5 is a block diagram of an apparatus according to an aspect to neuro-condition a breathing response using one or more sensors, a stimulator, a processor, a set of machine readable instructions that cause the processor to operate either in a training mode and a monitoring mode, and a physical switch that is read by the machine readable instructions to determine whether to cause the processor to operate in training mode or monitoring mode.

Apparatus 500 includes a sensor 510 operable to continuously sense a set of one or more physiological signals that change as breathing changes, a stimulator 520 operable to apply a physical stimulus, a processor 530 operable to receive the set of one or more physiological signals and further operable to trigger the stimulator to apply the physical stimulus, a set of machine readable instructions embodied in non-transitory memory 532 which cause the processor to operate in either a training mode or a monitoring mode, such that (i) when operating in the training mode, the machine readable instructions cause the processor to trigger the stimulator periodically at variable intervals determined by a predetermined set of rules, and such that (ii) when operating in the monitoring mode, the machine readable instructions cause the processor to detect a reduction in the rate of breathing to a predetermined rate value, or a cessation of breathing, and further cause the processor to trigger the stimulator to apply the physical stimulus when the reduction in the rate or the cessation of breathing is detected, and wherein the machine readable instructions are further operable to cause the processor to trigger the stimulator only when the on-skin sensor determines that the device is being worn on the body, and a physical switch 540 with two or more states, wherein one of the states represents the training mode and another of the states represents the monitoring mode, and wherein the machine readable instructions are further operable to read the state of the physical switch, determine when the state is changing, and further operable to cause the processor to operate in the training mode when the switch is in the state representing the training mode and to operate in the monitoring mode when the switch is in the state representing the monitoring mode.

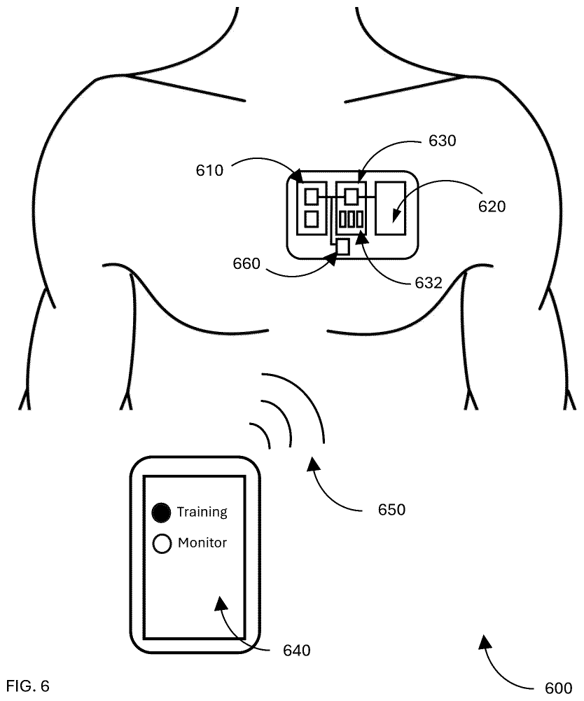

FIG. 6 is a block diagram of an apparatus according to an aspect to neuro-condition a breathing response using one or more sensors, a stimulator, a processor, a set of machine readable instructions that cause the processor to operate either in a training mode and a monitoring mode, and a communication system to receive a message representing either the training mode or the monitoring mode, and further operable to notify an external device when the stimulator is triggered when operating in monitoring mode.

Apparatus 600 includes a sensor 610 operable to continuously sense a set of one or more physiological signals that change as breathing changes, a stimulator 620 operable to apply a physical stimulus, a processor 630 operable to receive the set of one or more physiological signals and further operable to trigger the stimulator to apply the physical stimulus, a set of machine readable instructions embodied in non-transitory memory 632 which cause the processor to operate in either a training mode or a monitoring mode, such that (i) when operating in the training mode, the machine readable instructions cause the processor to trigger the stimulator periodically at variable intervals determined by a predetermined set of rules, and such that (ii) when operating in the monitoring mode, the machine readable instructions cause the processor to detect a reduction in the rate of breathing to a predetermined rate value, or a cessation of breathing, and further cause the processor to trigger the stimulator to apply the physical stimulus when the reduction in the rate or the cessation of breathing is detected, and wherein the machine readable instructions are further operable to cause the processor to trigger the stimulator only when the on-skin sensor determines that the device is being worn on the body, and a communication system 660, operable to send a notification to an external device or system when the stimulator is triggered by the processor when operating in monitoring mode.

In some aspects, the communication system 660 is further operable to receive a message having a representation of a desired state, limited to being either the training mode or the monitoring mode from an external user interface 640, and the communication system 660 is further operable to instruct the machine readable instructions to cause the processor to operate in the desired state. In some aspects, the external user interface 640 communicates with the communication system 660 wirelessly through the use of radio frequency (RF) waves 650.

### Method Aspects

In the previous section, apparatus of the operation of an aspect was described. In this section, the particular methods performed by usage of such an aspect are described by reference to a series of flowcharts.

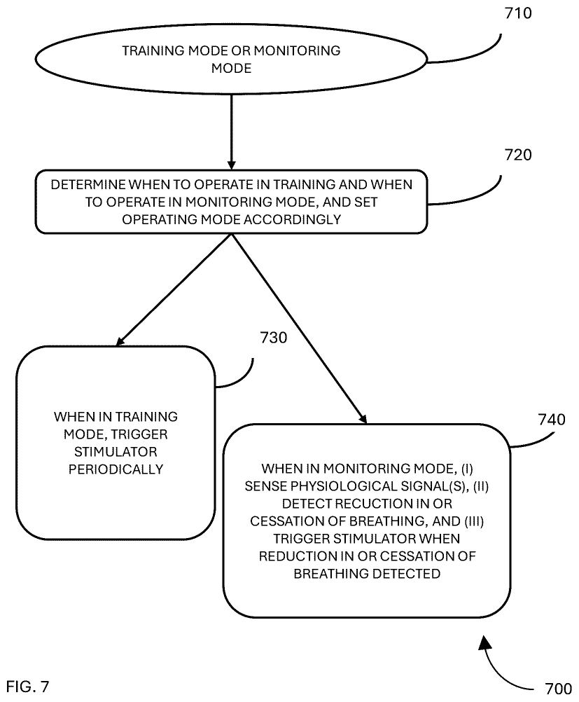

FIG. 7 is a flowchart of method 700 for neuro-conditioning a breathing response. Method 700 solves the need in the art to neuro-condition a breathing response by operating in a training mode and periodically triggering a stimulator. Method 700 also solves the need in the art to trigger a breathing response when needed by operating in a monitoring mode and sensing physiological signals that change as breathing changes, detecting a reduction in breathing or a cessation of breathing, and triggering the stimulator when the reduction in breathing or the cessation of breathing is detected.

Method 700 includes the operating mode, either training mode or monitoring mode 710, determining when to operate in training mode and when to operate in monitoring mode and setting the operating mode accordingly 720, and when operating in training mode, triggering a stimulator to periodically apply a physical stimulus 730, and when operating in monitoring mode, sensing one or more physiological signals that change as breathing changes, detecting a reduction in breathing or a cessation of breathing based on the sensed physiological signals, and triggering the stimulator to apply the physical stimulus when the reduction in breathing or the cessation of breathing is detected 740.

In some aspects, the physiological signals may include a plethysmography signal acquired by a PPG sensor, a movement signal, including chest movement signals, acquired by an inertial sensor such as an accelerometer or gyroscope, an airflow signal acquired by a gauge, an auscultation signal acquired by a microphone or other auditory sensor, and an end tidal carbon dioxide signal acquired by a CO₂ sensor. In other aspects, the stimulator may include an actuator, a vibration motor, a temperature stimulator, an electrical stimulator, or an audio stimulator.

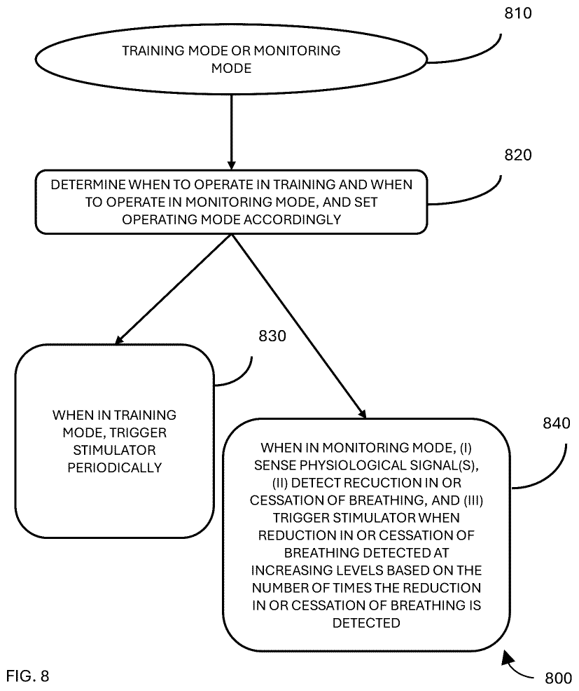

FIG. 8 is a flowchart of a method 800 to neuro-condition a breathing response by determining when to operate in a training and when to operate in a monitoring mode, setting the operating mode, and periodically triggering a stimulator when in training mode, and sensing physiological signals, detecting reduction or cessation of breathing, and triggering the stimulator at increasing levels based on the number of times the reduction or cessation of breathing is detected when in monitoring mode.

Method 800 includes the operating mode, either training mode or monitoring mode 810, determining when to operate in training mode and when to operate in monitoring mode and setting the operating mode accordingly 820, and when operating in training mode, triggering a stimulator to periodically apply a physical stimulus 830, and when operating in monitoring mode, sensing one or more physiological signals that change as breathing changes, detecting a reduction in breathing or a cessation of breathing based on the sensed physiological signals, and triggering the stimulator to apply the physical stimulus when the reduction in breathing or the cessation of breathing is detected at increasing levels based on the number of times the reduction in or cessation of breathing is detected 840.

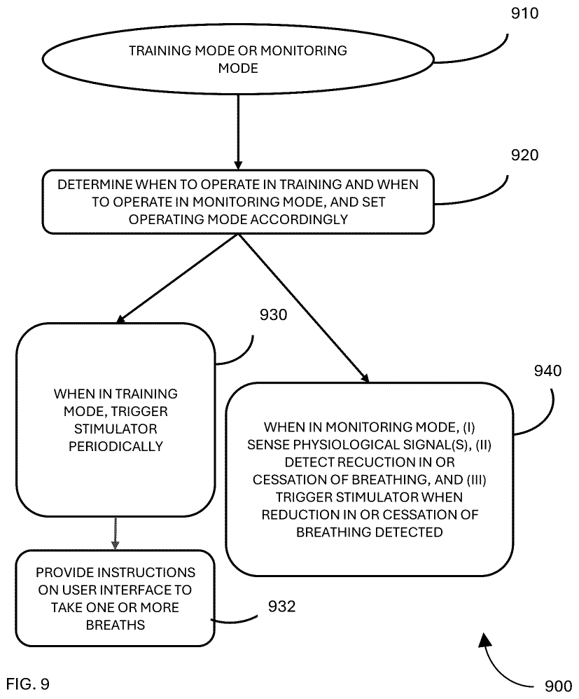

FIG. 9 is a flowchart of a method 900 to neuro-condition a breathing response by determining when to operate in a training and when to operate in a monitoring mode, setting the operating mode, and periodically triggering a stimulator and providing instructions on a user interface to take one or more breaths when in training mode, and sensing physiological signals, detecting reduction or cessation of breathing, and triggering the stimulator when in monitoring mode.

Method 900 includes the operating mode, either training mode or monitoring mode 910, determining when to operate in training mode and when to operate in monitoring mode and setting the operating mode accordingly 920, and when operating in training mode, triggering a stimulator to periodically apply a physical stimulus 930, and also providing instructions to take one or more breaths on a user interface 932 when the stimulus is felt, and when operating in monitoring mode, sensing one or more physiological signals that change as breathing changes, detecting a reduction in breathing or a cessation of breathing based on the sensed physiological signals, and triggering the stimulator to apply the physical stimulus when the reduction in breathing or the cessation of breathing is detected 940.

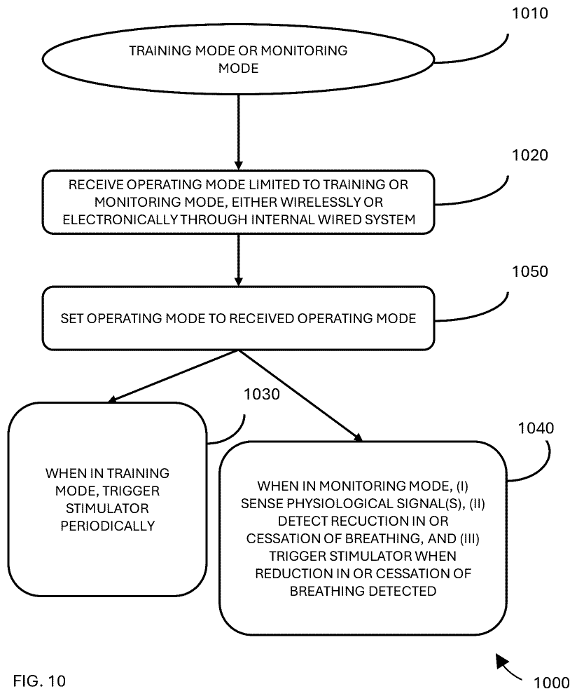

FIG. 10 is a flowchart of a method 1000 to neuro-condition a breathing response by receiving an operating mode, limited to training or monitoring modes, either wirelessly or electronically through an internal wired system, selecting to operate in a training or monitoring mode, and periodically triggering a stimulator when in training mode, and sensing physiological signals, detecting reduction or cessation of breathing, and triggering the stimulator when in monitoring mode.

Method 1000 includes the operating mode, either training mode or monitoring mode 1010, receiving a signal representing a desired operating mode limited to being either a training mode or a monitoring mode 1020, setting the operating mode to the received operating mode 1050, and when operating in training mode, triggering a stimulator to periodically apply a physical stimulus 1030, and when operating in monitoring mode, sensing one or more physiological signals that change as breathing changes, detecting a reduction in breathing or a cessation of breathing based on the sensed physiological signals, and triggering the stimulator to apply the physical stimulus when the reduction in breathing or the cessation of breathing is detected 1040. In some aspects, the signal representing the desired operating mode is received wirelessly from an external system. In other aspects, the signal representing the desired operating mode is received electronically via an internal wired system.

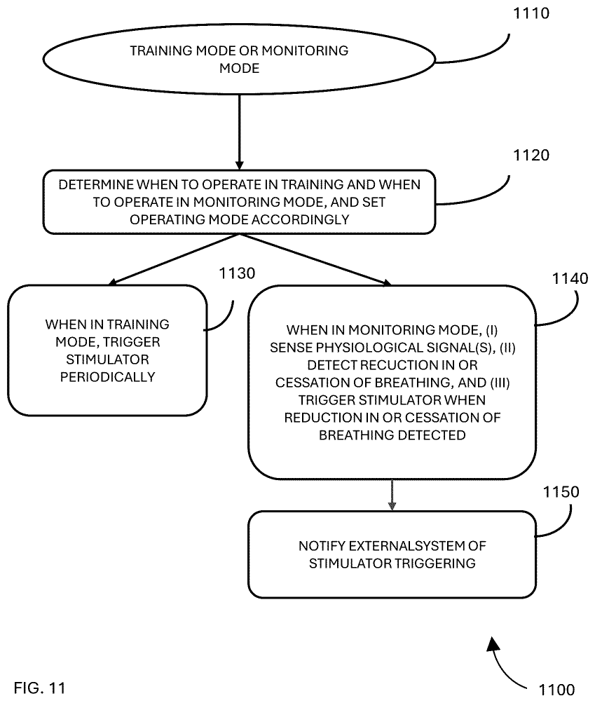

FIG. 11 is a flowchart of a method 1100 to neuro-condition a breathing response by determining when to operate in a training and when to operate in a monitoring mode, setting the operating mode, and periodically triggering a stimulator when in training mode, and sensing physiological signals, detecting reduction or cessation of breathing, triggering the stimulator, and notifying an external system of the triggering of the stimulator when in monitoring mode.

Method 1100 includes the operating mode, either training mode or monitoring mode 1110, determining when to operate in training mode and when to operate in monitoring mode and setting the operating mode accordingly 1120, and when operating in training mode, triggering a stimulator to periodically apply a physical stimulus 1130, and when operating in monitoring mode, sensing one or more physiological signals that change as breathing changes, detecting a reduction in breathing or a cessation of breathing based on the sensed physiological signals, and triggering the stimulator to apply the physical stimulus when the reduction in breathing or the cessation of breathing is detected 1140, and further to notify an external system when the stimulator is triggered when operating in monitoring mode.

### CONCLUSION

A system to establish neuro conditioning of a breathing response to a stimulus when breathing rate reduces or ceases is described. Although specific aspects are illustrated and described herein, it will be appreciated by those of ordinary skill in the art that any arrangement which is calculated to achieve the same purpose may be substituted for the specific aspects shown. This application is intended to cover any adaptations or variations. For example, although described in terms of body worn sensors to sense one or more physiological signals related to breathing, one of ordinary skill in the art will appreciate that implementations can be made using any other system capable of measuring changes in breathing rate or detecting cessation of breathing. Additionally, although described in terms of a stimulator applying a physical stimulus to the user, one of ordinary skill in the art will appreciate that implementations can also be made wherein the physical stimulus is applied by other means, for example through an external system. Further, also described in terms of a training mode and a monitoring mode, one of ordinary skill in the art will appreciated that more or fewer modes may be used to establish the neuro conditioning process.

In particular, one of skill in the art will readily appreciate that the names of the methods and apparatus are not intended to limit aspects. Furthermore, additional methods and apparatus can be added to the components, functions can be rearranged among the components, and new components to correspond to future enhancements and physical devices used in aspects can be introduced without departing from the scope of aspects. One of skill in the art will readily recognize that aspects are applicable to future types of sensors that sense other kinds of physiological signals that are correlated with breathing, and future types of stimulators that apply different kinds of physical stimuli.

The terminology used in this application is meant to include all systems to neuro-condition a breathing response and thereby elicit a breathing response upon reduction in or cessation of breathing, and alternate technologies which provide the same functionality as described herein.

What is claimed:

1. A body worn, breathing response neuro-conditioning device comprising:
a sensor operable to continuously sense a set of one or more physiological signals that change as breathing changes;
a stimulator operable to apply a physical stimulus;
a processor operable to receive the set of one or more physiological signals and further operable to trigger the stimulator to apply the physical stimulus; and
a set of machine readable instructions embodied in non-transitory memory which cause the processor to operate in either a training mode or a monitoring mode, such that (i) when operating in the training mode, the machine readable instructions cause the processor to trigger the stimulator periodically at variable intervals determined by a predetermined set of rules, and such that (ii) when operating in the monitoring mode, the machine readable instructions cause the processor to detect a reduction in the rate of breathing to a predetermined rate value, or a cessation of breathing, and further cause the processor to trigger the stimulator to apply the physical stimulus when the reduction in the rate or the cessation of breathing is detected.

2. The neuro-conditioning device of claim 1 wherein the one or more physiological signals are selected from the set comprising a photoplethysmography signal, a movement signal, an airflow signal, an auscultation signal, and an end tidal carbon dioxide signal.

3. The neuro-conditioning device of claim 1 wherein the stimulator is selected from the set comprising an actuator, a vibration motor, a temperature stimulator, an electrical stimulator, or an audio stimulator.

4. The neuro-conditioning device of claim 1 wherein the stimulator is further operable to apply the physical stimulus at varying amplitudes, and wherein when the machine readable instructions further cause the processor, when operating in the monitoring mode, to trigger the stimulator to apply the physical stimulus at increasing amplitudes based on the number of times the reduction in the rate or the cessation of breathing is detected.

5. The neuro-conditioning device of claim 1 further comprising an on-skin sensor to continuously determine when the device is being worn on the body and when it is not on the body.

6. The neuro-conditioning device of claim 5 wherein the machine readable instructions are further operable to cause the processor to trigger the stimulator only when the on-skin sensor determines that the device is being worn on the body.

7. The neuro-conditioning device of claim 1 further comprising a user interface with user instructions to take one or more breaths when the physical stimulus is felt when the processor is operating in the training mode.

8. The neuro-conditioning device of claim 1 further comprising a physical switch with two or more states, wherein one of the states represents the training mode and another of the states represents the monitoring mode, and wherein the machine readable instructions are further operable to read the state of the physical switch, determine when the state is changing, and further operable to cause the processor to operate in the training mode when the switch is in the state representing the training mode and to operate in the monitoring mode when the switch is in the state representing the monitoring mode.

9. The neuro-conditioning device of claim 1 further comprising a communication system operable to send a notification to an external device when the stimulator is triggered by the processor when operating in monitoring mode.

10. The neuro-conditioning device of claim 9 wherein the communication system is further operable to receive a message having a representation of a desired state, limited to being either the training mode or the monitoring mode, that instructs the machine readable instructions to cause the processor to operate in the desired state.

11. A method for conditioning a breathing response comprising:
determining when to operate in a training mode and when to operate in a monitoring mode;
when operating in the training mode, triggering a stimulator periodically at variable intervals determined by a predetermined set of rules, to apply a stimulus; and
when operating in the monitoring mode: (i) sensing a set of one or more physiological signals that change as breathing changes, (ii) detecting a reduction in the rate of breathing or a cessation of breathing from the set of one or more physiological signals, and (iii) triggering the stimulator to apply the physical stimulus when the reduction in the rate of breathing or the cessation of breathing is detected.

12. The method of claim 11 wherein the one or more physiological signals are selected from the set of a photoplethysmography signal, a movement signal, an airflow signal, an auscultation signal, and an end tidal carbon dioxide signal.

13. The method of claim 11 wherein the stimulator is selected from the set of an actuator, a vibration motor, a temperature stimulator, an electrical stimulator, or an audio stimulator.

14. The method of claim 11 wherein the stimulator is further operable to apply the physical stimulus at varying amplitudes, and wherein the step of triggering the stimulator to apply the physical stimulus when the reduction in the rate of breathing or the cessation of breathing is detected further comprises applying the physical stimulus at increasing amplitudes based on the number of times the reduction in the rate of breathing or the cessation of breathing is detected.

15. The method of claim 11 wherein the step of triggering the stimulator periodically at variable intervals determined by a predetermined set of rules, to apply a stimulus further comprises providing an instruction to take one or more breaths when the stimulus is felt.

16. The method claim 11 wherein the step of determining whether to operate in a training mode or in a monitoring mode further comprises receiving a signal representing a desired mode, wherein the desired mode is limited to either training mode or monitoring mode, and selecting to operate in training mode or monitoring mode based on the received desired mode.

17. The method of claim 16 wherein the signal to operate in the desired mode is received either wirelessly from an external system, or electronically via an internal, wired system.

18. The method of claim 11 further comprising notifying an external system when the stimulator is triggered when operating in monitoring mode.
